(19) United States
(12) Patent Application Publication
(10) Pub. No.: US 2019/0128981 A1
(43) Pub. Date: May 2, 2019

(54) NON-INVASIVE IMAGING METHOD FOR EARLY DETECTION AND MAPPING THE SEVERITY OF DISEASES BY USING CEST MRI

(71) Applicant: Ishtar Faisal Algburi, London (CA)

(72) Inventor: Ishtar Faisal Algburi, London (CA)

(73) Assignee: North America Imaging Developer, LLC, Detroit, MI (US)

(21) Appl. No.: 16/172,502

(22) Filed: Oct. 26, 2018

Related U.S. Application Data
(60) Provisional application No. 62/578,466, filed on Oct. 29, 2017.

(51) Int. Cl. G01R 33/48; G01R 33/56; A61K 38/17

(52) U.S. Cl. CPC G01R 33/4828; G01R 33/5601; G01N 2800/2821; G01R 33/5605; A61K 38/1716

(57) ABSTRACT

A non-invasive CEST MRI imaging method is disclosed for early detection and mapping the severity of diseases by using MRI. The endogenous magnetic resonance image (MRI) contrast of the biological tissue can rely on the endogenous protons of the proteins and peptides as a source of the contrast, such as hydroxyl, amine, and amide protons, and thereby provide imaging of the accumulation of amyloid beta, accumulation of neurofibrillary tangles, aggregation proteins and peptides, the hypoxia in cancer and non-cancer tissue, the tissues atrophy, distinguish the edema from the tumor, determine tumor boundary, monitor response of tumor to treatment and detect lower grade tumor by using endogenous protons contrast via CEST MRI. The difference in CEST images signals is used to detect and map severity of the diseases and predict response to treatment. The method works without contrast agents or tracers.

### CLAIM OF BENEFIT TO PRIOR APPLICATION

This application claims benefit to U.S. Provisional Patent Application 62/578,466, entitled “Non-invasive new imaging technique for early detection and mapping the severity of diseases using MRI in vitro and in vivo,” filed Oct. 29, 2017. The U.S. Provisional Patent Application 62/578,466 is incorporated herein by reference.

### BACKGROUND

Embodiments of the invention described in this specification relate generally to disease detection by imaging, and more particularly, to a non-invasive imaging method for early detection and severity mapping of diseases by using chemical exchange saturation transfer (“CEST”) magnetic resonance imaging (“MRI”).

Positron emission tomography (PET) scanners are used for detection and mapping amyloid beta plaques, and neurofibrillary tangles in the brains of living people. PET scanners use radiotracers (“tracers”) and/or contrast agents to detect disease. When injected into the bloodstream of a patient, tracers or contrast agents cross quickly into the brain, where they bind to amyloid plaques or neurofibrillary tangles to mark them with emissions of mild radioactivity. Amyloid beta imaging is highly useful to enable people to begin therapy early enough in time to avoid or to significantly delay the development of neurodegenerative diseases. Amyloid beta proteins are aggregated and accumulated at the extracellular space and form large accumulations of aggregation proteins, called amyloid beta plaques, which cause neuronal death. In the progression of the disease, amyloid beta plaques precede tau tangles, and both cause eventual neural loss, the accumulation of amyloid in the brain has been identified as an early biomarker of some diseases, such as Alzheimer's disease.

PET scanners also are used for mapping hypoxia in the tissue of living people. Hypoxia is a condition of insufficient oxygen to support metabolism which occurs when the vascular supply is interrupted. PET scanners map hypoxia also by tracers or contrast agents that are injected into the bloodstream. PET tracers have been used for the identification of hypoxia in living tissues and solid tumors by marking the hypoxia with emissions of mild radioactivity. Tumor hypoxia is the result of an inadequate supply of oxygen to tumor cells. Detection of hypoxia in tumors is of greater clinical relevance because tumor aggressiveness, metastatic spread, failure to achieve tumor treatment, and increased rate of recurrence are all associated with hypoxia. Tumor hypoxia increases resistance to radiotherapy and chemotherapy, resulting overall in poor clinical prognosis. Thus, in vivo measurement of tumor hypoxia could be helpful to identify patients with worse prognosis or patients who could benefit from appropriate treatments, such as radiation therapy or chemotherapy.

Brain atrophy reflects the destructive pathological processes in many diseases. The current methods for imaging of brain atrophy using standard MRI can detect brain atrophy at a late stage of the disease(s) causing the atrophy by measuring the changes of the ventricle volume of the brain. Most of the methods are sensitive to subtle changes in brain structures and have been applied to diseases as measures of whole-brain atrophy years after atrophy starts and the progression of the disease is increasing.

While many MRI imaging methods provide clear results, it is typically difficult to distinguish between edema, tumor, and tumor boundary by using current methods that use MRI, because increase the water in both, the edema and the tumor look bright on T₂-weighted image or dark on T₁-weighted image. Also, these MRI methods are unable to detect small metastasis tumor. In most cases, it helps to use a contrast agent, such as Gadolinium, to better identify the tumor and distinguish the edema from the tumor. However, Gadolinium is expensive and has many side effects on humans, especially to humans with kidney diseases.

Therefore, what is needed is a way to use CEST MRI imaging to detect and map the accumulation of amyloid beta, neurofibrillary tangles, and aggregation proteins and peptides by using endogenous protons of accumulated proteins to detect these proteins and peptides, and a way to image the hypoxia in cancer and non-cancer tissue, the atrophy and distinguish the edema from the tumor and determine tumor boundary very precisely by using endogenous protons contrast by MRI as opposed to PET scans, which are longer in time, not as safe as MRI, and provide lower resolution images than MRI, and to do so without the need to inject any contrast agents or tracers.

### BRIEF DESCRIPTION

Novel non-invasive imaging methods for early detection of disease and disease severity mapping using CEST MRI are disclosed. The non-invasive imaging methods for early detection of disease and disease severity mapping using CEST MRI include a non-invasive CEST MRI imaging method for early detection of disease and mapping of disease severity and a non-invasive CEST MRI amyloid beta plaque imaging method for early detection of amyloid beta plaques and mapping of amyloid beta plaque accumulation. In some embodiments, the non-invasive imaging methods for early detection of disease and disease severity mapping using CEST MRI are designed for imaging accumulation of amyloid beta, neurofibrillary tangles, and aggregation proteins and peptides by using endogenous protons of accumulated proteins to detect these proteins and peptides by way of CEST MRI. In some embodiments, the non-invasive imaging methods for early detection of disease and disease severity mapping using CEST MRI are also designed for imaging the hypoxia and the atrophy in cancer and non-cancer tissue, for distinguishing the edema from the tumor, and for determining tumor boundary very precisely by using endogenous protons contrast by MRI.

In some embodiments, the non-invasive CEST MRI imaging method for early detection of disease and mapping of disease severity includes a plurality of steps comprising (i) acquiring, by way of a magnetic resonance imaging (MRI) machine, a T₂-image as an anatomical image, (ii) acquiring, by way of the MRI machine, a CEST image at S_{outside} as a reference image, (iii) acquiring, by way of the MRI machine, a plurality of CEST images at S_{within}, (iv) normalizing signal intensities of the acquired CEST images (S_{outside} and S_{within}) to the reference image, (v) calculating a contrast difference in S (ΔS contrast=S_{outside}−S_{within} for each image in the plurality of CEST images at S_{within}), and (vi) detecting disease and mapping disease severity based on the calculated ΔS contrast. In some embodiments, the non-invasive CEST MRI imaging method for early detection of disease and mapping of disease severity measures a chemical shift of the reference image >10 ppm or <−10 ppm. For example, the non-invasive CEST MRI imaging method for early detection of disease and mapping of disease severity can use 11 ppm or 20 ppm downfield or use −11 ppm or −20 ppm upfield.

In some embodiments, the non-invasive CEST MRI amyloid beta plaque imaging method for early detection of amyloid beta plaques and mapping of amyloid beta plaque accumulation in a brain includes a plurality of steps comprising (i) acquiring, by way of an MRI machine, a T₂-image as an anatomical image of the brain, (ii) acquiring, by way of the MRI machine, a CEST reference image at S_{outside}=11 ppm, (iii) acquiring, by way of the MRI machine, a plurality of CEST images at S_{within}=3.5 ppm or 3.4 ppm, (iv) normalizing signal intensities of the acquired CEST images (S_{outside} and S_{within}) to the reference image at S_{outside}=11 ppm, (v) calculating a contrast difference in S (ΔS contrast=S_{outside}−S_{within} for each image in the plurality of CEST images at S_{within}), and (vi) detecting amyloid beta plaques and mapping amyloid beta plaque accumulation in the brain based on the calculated ΔS contrast.

The preceding Summary is intended to serve as a brief introduction to some embodiments of the invention. It is not meant to be an introduction or overview of all inventive subject matter disclosed in this specification. The Detailed Description that follows and the Drawings that are referred to in the Detailed Description will further describe the embodiments described in the Summary as well as other embodiments. Accordingly, to understand all the embodiments described by this document, a full review of the Summary, Detailed Description, and Drawings is needed. Moreover, the claimed subject matters are not to be limited by the illustrative details in the Summary, Detailed Description, and Drawings, but rather are to be defined by the appended claims, because the claimed subject matter can be embodied in other specific forms without departing from the spirit of the subject matter.

### BRIEF DESCRIPTION OF THE DRAWINGS

Having described the invention in general terms, reference is now made to the accompanying drawings, which are not necessarily drawn to scale, and wherein:

FIG. 1 conceptually illustrates a non-invasive CEST MRI imaging method for early detection of disease and mapping of disease severity in some embodiments.

FIG. 2 conceptually illustrates a non-invasive CEST MRI amyloid beta plaque imaging method for early detection of amyloid beta plaques and mapping of amyloid beta plaque accumulation in some embodiments.

FIG. 3 conceptually illustrates an electronic system with which some embodiments of the invention are implemented.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments of the invention include novel non-invasive imaging methods for early detection of disease and disease severity mapping using CEST MRI. The non-invasive imaging methods for early detection of disease and disease severity mapping using CEST MRI include a non-invasive CEST MRI imaging method for early detection of disease and mapping of disease severity and a non-invasive CEST MRI amyloid beta plaque imaging method for early detection of amyloid beta plaques and mapping of amyloid beta plaque accumulation. In some embodiments, the non-invasive imaging methods for early detection of disease and disease severity mapping using CEST MRI are designed for imaging accumulation of amyloid beta, neurofibrillary tangles, and aggregation proteins and peptides by using endogenous protons of accumulated proteins to detect these proteins and peptides by way of CEST MRI. In some embodiments, the non-invasive imaging methods for early detection of disease and disease severity mapping using CEST MRI are also designed for imaging the hypoxia and the atrophy in cancer and non-cancer tissue, for distinguishing the edema from the tumor, and for determining tumor boundary very precisely by using endogenous protons contrast by MRI.

The non-invasive CEST MRI imaging methods for early detection of disease and disease severity mapping of the present disclosure are based on the use of CEST MRI, not PET and without contrast agents or radio tracers. The endogenous magnetic resonance image (MRI) contrast of the biological tissue can rely on the endogenous protons of the proteins and peptides as a source of the contrast, such as hydroxyl, amine, and amide protons, and thereby provide imaging of the accumulation of amyloid beta, accumulation of neurofibrillary tangles, aggregation proteins and peptides, the hypoxia in cancer and non-cancer tissue, the tissues atrophy, distinguish the edema from the tumor, determine tumor boundary, monitor response of tumor to treatment and detect lower grade tumor by using endogenous protons contrast via CEST MRI. The difference in CEST images signals is used to detect and map severity of the diseases and predict response to treatment.

The non-invasive imaging methods for early detection of disease and disease severity mapping using CEST MRI of the present disclosure may produce or result in the following elements. This list of possible constituent elements is intended to be exemplary only and it is not intended that this list be used to limit the non-invasive imaging methods for early detection of disease and disease severity mapping using CEST MRI of the present application to just these elements. Persons having ordinary skill in the art relevant to the present disclosure may understand there to be equivalent elements that may be substituted within the present disclosure without changing the essential function or operation of the non-invasive imaging methods for early detection of disease and disease severity mapping using CEST MRI.

1. CEST image-(the reference image at S_{outside})

2. CEST images (multiple images at S_{within})

3. Difference in magnetization (ΔS contrast images=S_{outside}−S_{within})

4. Resulting in detection of disease(s) and mapping of severity of disease(s)

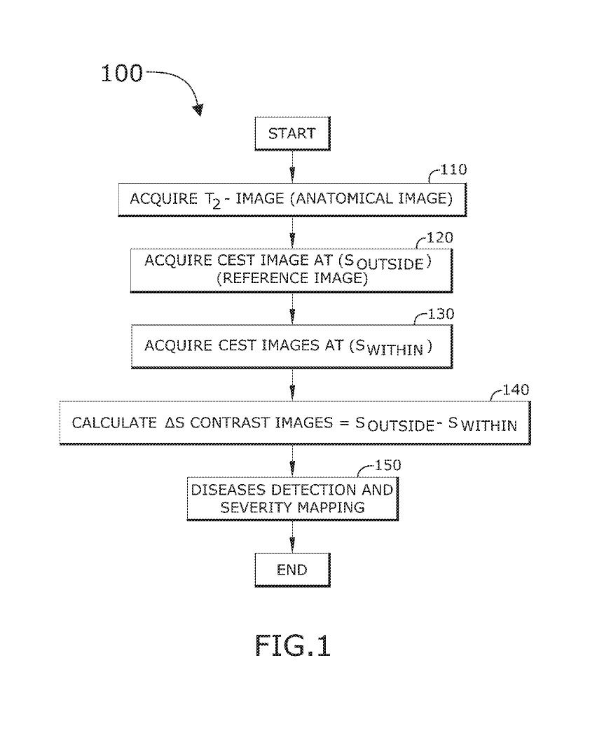

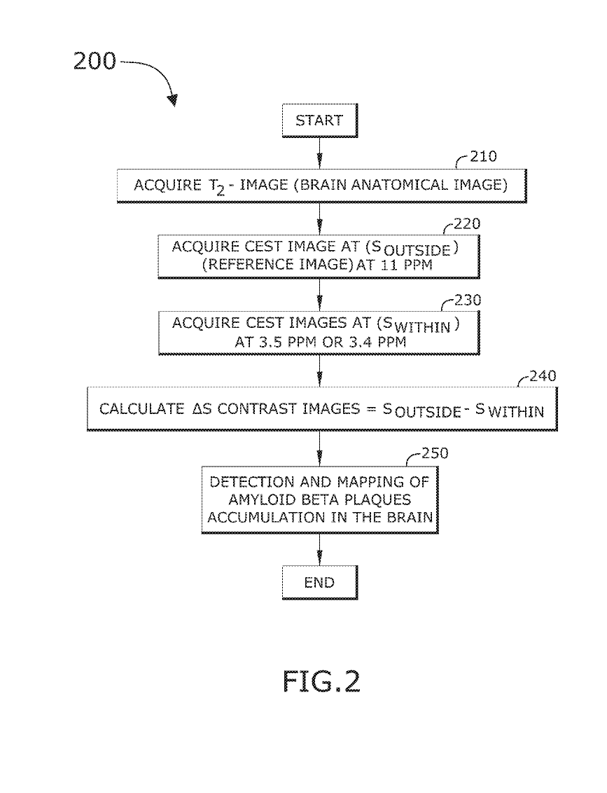

The non-invasive imaging methods for early detection of disease and disease severity mapping using CEST MRI of the present disclosure generally work by a sequence of actions or operation (steps). While the non-invasive imaging methods for early detection of disease and disease severity mapping using CEST MRI are described further below by reference to FIGS. 1 and 2, the methods are generally detailed as follows.

Step 1: Acquiring anatomical T₂-image(s) of specific areas of the brain which are areas of focus as expected under the effects of certain particular diseases. For instance, specific areas of the brain include the hippocampus and the cortex in Alzheimer's disease.

Step 2: Acquiring a CEST image (reference image, S_{outside}) at a specific chemical shift and at a signal frequency outside the range of frequency that decreases magnetization of related proteins.

Step 3 (or contemporaneously with Step 2): Acquiring CEST images at specific chemical shifts and at frequencies that decrease the magnetization of the proteins (S_{within}). The CEST chemical shift correction is preferred to be done when B₀ variation is larger than 8%. Also, all the CEST images (via signals intensities) are normalized to the reference image. For example, acquiring CEST images around 3.5 ppm for amide proton (between 3.1 ppm to 4 ppm with a step size of 0.1 ppm) as S_{within} images, and acquiring CEST images around 2.5 ppm for amine proton (between 1 ppm to 3 ppm with a step size of 0.1 ppm) as S_{within} images.

The non-invasive imaging methods for early detection of disease and disease severity mapping using CEST MRI of the present disclosure support most of the pulse sequences for CEST images. For example, one may use fast spin-echo (FSE) pulse sequence by using the following parameters: TR=3 s; TE=6.4 ms; FOV=212×190 mm²; matrix size=256×256; slice thickness=4.4 mm; turbo-spin-echo factor=45; and single slice acquisition. The RF saturation section includes a series of four block RF saturation pulses (200 ms duration each and 2 μT amplitude) at 3Tesla for the human.

To use the non-invasive imaging methods for early detection of disease and disease severity mapping using CEST MRI of the present disclosure, a person working on MRI (an MRI operator) can follow the general steps noted above (Step 1, Step 2, and Step 3). Thus, to obtain accurate disease detection and severity mapped results, the MRI operator drives the actions noted in the steps of the methods by control operations with respect to the CEST MRI machine, which captures the images, and by interaction with a computing device, which performs computations, thereby allowing the MRI operator to follow the general steps (Step 1, Step 2, and Step 3) to get the results.

The MRI operator may carry out more specific steps, such as those described below by reference to FIGS. 1 and 2. Furthermore, the MRI operator can produce ΔS images contrast by using MATLAB (MathWorks) on a computing device and thereby make determinations of diseases that may be present. For instance, since ΔS increases as accumulation or the concentration of the proteins increases, ΔS can be used to identify the severity of neurodegenerative diseases that involve aggregation proteins amyloidogenesis. The MRI operator can detect many such diseases and map the severity by adhering to the following observations and rules: ΔS contrast can detect and map the distribution of amyloid beta plaques, aggregation proteins in the areas or the structures such as the hippocampus and the cortex that are susceptible to amyloid beta plaques, aggregation proteins, and neurofibrillary tangles. ΔS contrast is higher for the tumor tissue compared to normal tissue due to an increase of the exchange rate of the protons. The hypoxic tumor tissue has higher ΔS than non hypoxic tumor tissue because the hypoxic tumor is more alkaline than non hypoxic tumor. The normal tissue (ΔS is lower) than tumor tissue (ΔS is higher), and it is easy to identify the tumor boundary from the edema (ΔS is the lowest value for the edema) very precisely. In non-cancer tissue, ΔS is lower for hypoxic tissue compared to normal tissue by using chemical shift that decreases magnetization of amide (between 3.1 ppm to 4 ppm for detection of hypoxia for non-cancer tissue) contrast as S_{within} but ΔS is higher compared to normal tissue if using chemical shift that decreases magnetization of amine proton (between 1 ppm to 3 ppm for detection of hypoxia for non-cancer tissue). The tissue under atrophy has ΔS little higher than ventricle or water areas depend on the severity of the atrophy (as the atrophy is severe ΔS will be close to the ventricles and the water area).

Several more detailed embodiments are described in the sections below. Section I describes a variety of applications for non-invasive imaging for early detection of disease and mapping of disease severity using CEST MRI without use of contrast agents or tracers. Section II describes some non-invasive imaging methods for early detection of disease and disease severity mapping using CEST MRI. Section III describes an electronic system that implements some embodiments of the non-invasive imaging methods described herein.

I. Non-Invasive Imaging for Early Detection of Disease and Mapping of Disease Severity Using CEST MRI without Contrast Agents or Radio Tracers

The endogenous protons such as hydroxyl, amine, and amide protons of accumulated amyloid beta plaques, aggregation proteins, and neurofibrillary tangles can be used as endogenous contrast to detect amyloid beta plaques and aggregation proteins of neurodegenerative diseases. From this contrast, it is possible to map the distribution and accumulation of these proteins and peptides in brains or any other parts of the body. Any method of decreasing magnetization of protons of these proteins and peptides by MRI can be used to detect these proteins such as chemical exchange saturation transfer (“CEST”) to detect amyloid beta plaques, neurofibrillary tangles, and aggregation proteins such as in neurodegenerative diseases that involve amyloidogenesis.

The endogenous hydroxyl, amine, and amide protons of tissues also can be used as endogenous contrast to detect the hypoxia in cancer and non-cancer tissue and the tissue atrophy and to distinguish the water, the edema from the tumor, and determine tumor boundary very precisely by using MRI. From this contrast, it is also possible to map the response of tumor to the treatment, hypoxia, atrophy, and edema, and to detect the tumor. Any method of decreasing magnetization of protons of these proteins and peptides by MRI can be used to detect the diseases such as the hypoxia, atrophy, edema, and tumor, and to monitor response of the tumor to the treatment by using CEST MRI.

The chemical shift of water is 0 parts per million (“ppm”). The decreased magnetization of amyloid beta plaques, aggregation proteins, and neurofibrillary tangles proteins starts from chemical shift (0.05 ppm to 10 ppm) downfield and from (−0.05 ppm to −10 ppm) upfield. The maximum decrease of the magnetization of amyloid beta, neurofibrillary tangles, and aggregation proteins approximately (between 1 ppm and 5 ppm) downfield and approximately (between −1 ppm to −5 ppm) upfield. Using pulse sequence, saturation power, and duration time that optimize amine and amide protons exchange can be used to detect these proteins and peptides by acquiring the T₂ image and CEST images (which is further described below, by reference to FIGS. 1 and 2).

The decrease in magnetization of cancer proteins also starts from chemical shift (0.05 ppm to 10 ppm) downfield and from (−0.05 ppm to −10 ppm) upfield. Also, saturation power and duration time that optimize hydroxyl, amine, and amide protons exchange can be used to detect these proteins and peptides (which is further described below, by reference to FIG. 1). The general method is carried out by first acquiring an anatomical T₂-image of a specific area that is the expectation under the effect of these diseases. Second, the method acquires CEST images at specific chemical shift at a frequency outside the range of frequency that decreases magnetization of these proteins (as a reference image). Third, the method then acquires (multiple) CEST images at specific chemical shifts, at frequencies that decrease the magnetization of these proteins. The CEST chemical shift correction preferred to be done if (B₀ variation is larger than 8%). Also, all the CEST images (the signal intensities) should be normalized to the reference image. Then the method determines the difference between the magnetization of reference CEST image (an image at saturation chemical shift outside the chemical shift range of decreasing magnetization of these proteins) and CEST image (an image at saturation chemical shift that decreases magnetization of these proteins). This difference in magnetization (ΔS) of CEST images can be used to detect and map the distribution and accumulation of the amyloid beta plaques, aggregation proteins and neurofibrillary tangles, the hypoxia, atrophy, edema, and tumor, and to monitor the response of the tumor to the treatment and identify the severity of the diseases.

The difference in magnetization (ΔS) of CEST images=magnetization at saturation chemical shift outside the range of decreasing magnetization of these proteins (S_{outside})−magnetization at saturation chemical shift within the range of decrease magnetization of these proteins (S_{within}) between (0.05 ppm to 10 ppm or −0.05 ppm to −10 ppm). Also, all CEST images should be normalized to the reference CEST image, (the chemical shift of the reference image >10 ppm and <−10 ppm) such as using 11 ppm or 20 ppm downfield or using −11 ppm or −20 ppm upfield where there are no metabolites at these chemical shifts.

This difference in magnetization can be expressed as: ΔS=S_{outside}−S_{within}

The difference in magnetization (ΔS) can be used for mapping the brain or any part in the body under amyloid beta plaques, aggregation proteins, and neurofibrillary tangles. Amyloid beta plaques, aggregation proteins, and neurofibrillary tangles tissues have higher contents of proteins compared to surrounding normal tissue. These proteins contain high concentrations of protons (such as amide and amine protons) and the exchange rate of amine and amide protons is increased in the area where amyloid beta plaques, aggregation proteins, and neurofibrillary tangles are accumulated in the diseases (such as neurodegenerative diseases) compared to the normal tissue. The difference in magnetization, ΔS, is higher for the area that is susceptible to amyloid beta plaques, aggregation proteins, and neurofibrillary tangles tissues compared to normal surrounding tissue and ΔS is increased as accumulation or the concentration of these proteins is increased. The difference in magnetization, ΔS, can also be used to identify the severity of neurodegenerative diseases that involve aggregation proteins amyloidogenesis. Specifically, ΔS contrast can detect and map the distribution of amyloid beta plaques, aggregation proteins in the areas or the structures such as the hippocampus and the cortex that are susceptible to amyloid beta plaques, aggregation proteins, and neurofibrillary tangles easily and can be used as early detection of neurodegenerative diseases that involve aggregation proteins amyloidogenesis when the aggregation proteins and amyloid protein and tangles accumulation start before many years from neurodegenerative diseases symptoms appear.

The ΔS contrast can be used to mapping and detection the hypoxia in cancer and non-cancer tissue, the atrophy, and distinguishing of the edema from the tumor, and the determination of the tumor boundary very precisely. Tumor tissue has a higher content of protein (amine and amide protons) compared to normal tissue and the exchange rate of amine and amide protons is increased in tumor tissue compared to normal tissue. As such, the ΔS contrast is higher for the tumor tissue compared to normal tissue due to an increase of the exchange rate of the protons. The hypoxic tumor tissue has higher ΔS than non-hypoxic tumor tissue because the hypoxic tumor is more alkaline than non-hypoxic tumor. Therefore, increasing the exchange rate of amide protons of the hypoxic tumor and moving away from the tumor boundary toward the tumor core results in the ΔS increasing. This refers to or is indicative of an increase of the hypoxia in the core of tumor tissue because of an increase in amide proton exchange rates in hypoxic-tumor tissue compared to non-hypoxic tumor tissue. From this comparison, it is easy to identify the hypoxia in tumor tissue. Also, it is possible to identify the normal tissue (where ΔS is lower) than tumor tissue (where ΔS is higher), and it is easy to identify the tumor boundary from the edema (where ΔS is the lowest value for the edema) very precisely.

In non-cancer tissue, the difference in magnetization (ΔS) is lower for hypoxic tissue compared to normal tissue when using a chemical shift that decreases magnetization of amide proton (i.e., between 3.1 ppm to 4 ppm for detection of hypoxia for non-cancer tissue) contrast as S_{within}. On the other hand, the ΔS is higher for hypoxic tissue compared to normal tissue when using a chemical shift that decreases magnetization of amine proton (between 1 ppm to 3 ppm for detection of hypoxia for non-cancer tissue) contrast as S_{within} because the with a decrease of exchange rate of amide protons of hypoxic tissue compared to normal tissue, as moving toward core of the hypoxic tissue, the ΔS is lower, which is indicative of hypoxic tissue core because the decrease amide proton exchange rate and increase amine proton exchange rate in hypoxic tissue compared to normal tissue from this contrast it is possible to identify the hypoxic tissue in non-tumor tissue by using amide or amine protons.

The decreased magnetization of atrophy proteins also starts from a chemical shift (0.05 ppm to 10 ppm) downfield and (−0.05 ppm to −10 ppm) upfield. In the atrophy, such as brain atrophy, ΔS is lowest at ventricles and the water area. The tissue under atrophy has ΔS a little higher than the ventricles or the water areas depending on the severity of the atrophy (when the atrophy is severe, ΔS will be close to the ventricles and the water area) because the ventricles have less protein content and therefore, fewer protons (such as amine and amide protons) and, accordingly, lower exchange rates compared to surrounding tissue. The atrophy tissue has higher protein content than the ventricles or water areas. However, the atrophy tissue has lower protein content than normal tissue. Therefore, the atrophy tissue has higher proton exchange rates than the ventricles and much less than normal tissue. ΔS for the atrophy tissue is much lower than normal tissue and as the severity of the atrophy increases the ΔS decreases, so by using a chemical shift that decreases magnetization of amide (between 3.1 ppm to 4 ppm) as S_{within}, or by using chemical shift that decreases magnetization of amine proton (between 1 ppm to 3 ppm) as S_{within}, from this contrast (ΔS) it is easy to detect the atrophy and identify the severity of the atrophy in the different parts of the body. This novel imaging technique can be used for early detection of atrophy years before structural tissue changes start, such as the brain structures. This is a great improvement over the existing practice, presently used in hospitals and medical imaging centers, of measuring the tissue structure change years after starting the tissue atrophy.

The difference in magnetization (ΔS) contrast can also be used to detect lower grade tumor that cannot be detected or distinguished by other methods. Furthermore, the ΔS contrast can distinguish the higher-grade tumor from lower-grade tumor, where ΔS increases as the tumor grade and the aggressiveness increases when using chemical shift that decreases magnetization of amide proton with max contrast (between 3.1 ppm to 4 ppm) as S_{within}. Also, ΔS contrast can monitor the response of tumor to treatment, such as chemotherapy and radiation therapy, in most types of cancer if the tumor response to the treatment causes a decrease in ΔS compared to ΔS before the treatment (degree of tumor responsiveness to the treatment depends on the magnitude of decrease in ΔS value after the treatment) when using a chemical shift that decreases magnetization of amide proton with max contrast (between 3.1 ppm to 4 ppm) as S_{within}, because of a decrease in amide protons exchange rate. Also, the ΔS is increased, after responding to the treatment of the tumor as being cancer, by using a chemical shift that decreases magnetization of amine proton with max contrast (between 1 ppm to 3 ppm) as S_{within}, because of an increase in amine protons exchange rate. Therefore, using ΔS value can help in prediction of the respond of cancer to the different treatments.

### II. Non-Invasive Imaging Methods for Early Detection of Disease and Disease Severity Mapping Using CEST MRI

In some embodiments, the non-invasive CEST MRI imaging method for early detection of disease and mapping of disease severity includes a plurality of steps comprising (i) acquiring, by way of a magnetic resonance imaging (MRI) machine, a T₂-image as an anatomical image, (ii) acquiring, by way of the MRI machine, a CEST image at S_{outside} as a reference image, (iii) acquiring, by way of the MRI machine, a plurality of CEST images at S_{within}, (iv) normalizing signal intensities of the acquired CEST images (S_{outside} and S_{within}) to the reference image, (v) calculating a contrast difference in S (ΔS contrast=S_{outside}−S_{within} for each image in the plurality of CEST images at S_{within}), and (vi) detecting disease and mapping disease severity based on the calculated ΔS contrast. In some embodiments, the non-invasive CEST MRI imaging method for early detection of disease and mapping of disease severity measures a chemical shift of the reference image >10 ppm or <−10 ppm. For example, the non-invasive CEST MRI imaging method for early detection of disease and mapping of disease severity can use 11 ppm or 20 ppm downfield or use −11 ppm or −20 ppm upfield.

By way of example, FIG. 1 conceptually illustrates a non-invasive CEST MRI imaging method for early detection of disease and mapping of disease severity 100. The non-invasive CEST MRI imaging method for early detection of disease and mapping of disease severity 100 may be performed by an MRI operator in connection with a CEST MRI machine for image acquisition and computing device for certain computation. As shown in this figure, the non-invasive CEST MRI imaging method for early detection of disease and mapping of disease severity 100 starts by acquiring a T₂-image as an anatomical image (at 110). In some embodiments, the CEST MRI machine captures the anatomical image of a human brain when the MRI operator issues an instruction or command to acquire the T₂-image for the anatomical image. Next, the non-invasive CEST MRI imaging method for early detection of disease and mapping of disease severity 100 continues to the next step of acquiring CEST image at S_{outside} as a reference image (at 120). In some embodiments, the CEST MRI machine captures the reference image when instructed by the MRI operator to acquire the CEST image at S_{outside} as the reference image. After, or contemporaneously with, acquiring the reference image, the non-invasive CEST MRI imaging method for early detection of disease and mapping of disease severity 100 acquires CEST images at S_{within} (at 130). In some embodiments, the CEST MRI machine captures a plurality of CEST images at S_{within} when the MRI operator signals for the acquisition of images. If needed, the non-invasive CEST MRI imaging method for early detection of disease and mapping of disease severity 100 of some embodiments normalizes signal intensities of the acquired CEST images (S_{outside} and S_{within}) to the reference image.

Next, the non-invasive CEST MRI imaging method for early detection of disease and mapping of disease severity 100 calculates (at 140) the difference in magnetization (ΔS) of the CEST images, where ΔS=S_{outside}−S_{within}. In some embodiments, the calculation of ΔS is performed by the MRI operator interacting with a computing device and using MATLAB (MathWorks) to find the difference. Specifically, ΔS is calculated as the difference between magnetization at saturation chemical shift outside the range of decreasing magnetization of the proteins (S_{outside}) and magnetization at saturation chemical shift within the range of decrease magnetization of these proteins (S_{within}). This difference in magnetization is expressed as: ΔS=S_{outside}−S_{within}.

In some embodiments, the non-invasive CEST MRI imaging method for early detection of disease and mapping of disease severity 100 then detects diseases based on the results and performs disease severity mapping (at 150). The MRI operator can then see the results of detected diseases and their relative severity. Then the non-invasive CEST MRI imaging method for early detection of disease and mapping of disease severity 100 ends.

While the non-invasive CEST MRI imaging method for early detection of disease and mapping of disease severity 100 summarizes steps of a general process for disease detection and disease severity mapping, each type of disease noted above can be detected and mapped for severity by specific configurable settings (also as described above). One embodiment performs this for amyloid beta plaques and mapping the accumulation of plaques in relation to Alzheimer's disease detection. Specifically, in some embodiments, the non-invasive CEST MRI amyloid beta plaque imaging method for early detection of amyloid beta plaques and mapping of amyloid beta plaque accumulation in a brain includes a plurality of steps comprising (i) acquiring, by way of an MRI machine, a T₂-image as an anatomical image of the brain, (ii) acquiring, by way of the MRI machine, a CEST reference image at S_{outside}=11 ppm, (iii) acquiring, by way of the MRI machine, a plurality of CEST images at S_{within}=3.5 ppm or 3.4 ppm, (iv) normalizing signal intensities of the acquired CEST images (S_{outside} and S_{within}) to the reference image at S_{outside}=11 ppm, (v) calculating a contrast difference in S (ΔS contrast=S_{outside}−S_{within} for each image in the plurality of CEST images at S_{within}), and (vi) detecting amyloid beta plaques and mapping amyloid beta plaque accumulation in the brain based on the calculated ΔS contrast.

By way of example, FIG. 2 conceptually illustrates a non-invasive CEST MRI amyloid beta plaque imaging method for early detection of amyloid beta plaques and mapping of amyloid beta plaque accumulation 200. As shown in this figure, the non-invasive CEST MRI amyloid beta plaque imaging method for early detection of amyloid beta plaques and mapping of amyloid beta plaque accumulation 200 starts by acquiring (at 210) a T₂-image as an anatomical image of the brain of a person by way of an MRI machine. Next, the non-invasive CEST MRI amyloid beta plaque imaging method for early detection of amyloid beta plaques and mapping of amyloid beta plaque accumulation 200 acquires (at 220) the CEST reference image at S_{outside}=11 ppm (again, by way of the MRI machine). After acquiring the reference image, the non-invasive CEST MRI amyloid beta plaque imaging method for early detection of amyloid beta plaques and mapping of amyloid beta plaque accumulation 200 acquires (at 230) a plurality of CEST images at S_{within}=3.5 ppm or 3.4 ppm, by the MRI machine as operated by the MRI operator. If needed, the non-invasive CEST MRI amyloid beta plaque imaging method for early detection of amyloid beta plaques and mapping of amyloid beta plaque accumulation 200 of some embodiments normalizes signal intensities of the acquired CEST images (S_{outside} and S_{within}) to the reference image at S_{outside}=11 ppm.

After the specific CEST images are acquired and the signal intensities are normalized, then the non-invasive CEST MRI amyloid beta plaque imaging method for early detection of amyloid beta plaques and mapping of amyloid beta plaque accumulation 200 calculates (at 240) contrast difference in S (ΔS contrast=S_{outside}−S_{within} for each image in the plurality of CEST images at S_{within}). In some embodiments, the calculation of ΔS is performed by the MRI operator interacting with a computing device and using MATLAB (MathWorks) to find the difference. Specifically, ΔS is calculated as the difference between magnetization at saturation chemical shift outside the range of decreasing magnetization of the proteins (S_{outside}) and magnetization at saturation chemical shift within the range of decrease magnetization of these proteins (S_{within}) between (0.05 ppm to 10 ppm or −0.05 ppm to −10 ppm). Also, all CEST images should be normalized to the reference CEST image, (the chemical shift of the reference image >10 ppm and <−10 ppm) such as using 11 ppm or 20 ppm downfield or using −11 ppm or −20 ppm upfield where there are no metabolites at these chemical shifts.

In some embodiments, detection and mapping of amyloid beta plaques accumulation in the brain is performed (at 250) by the non-invasive CEST MRI amyloid beta plaque imaging method for early detection of amyloid beta plaques and mapping of amyloid beta plaque accumulation 200. Then the non-invasive CEST MRI amyloid beta plaque imaging method for early detection of amyloid beta plaques and mapping of amyloid beta plaque accumulation 200 ends.

While the example described above by reference to FIG. 2 pertains specifically to detection of amyloid beta plaque accumulation in the brain of a person who may have or soon have Alzheimer's disease, any of several sets of chemical shifts for various diseases (as described in detail above) can be applied to the general method described above by reference to FIG. 1 to calculate the ΔS contrast=S_{outside}−S_{within} in the detection of disease and in mapping the severity of the disease.

### III. Electronic System

Many of the above-described features and applications are implemented as software processes that are specified as a set of instructions recorded on a computer readable storage medium (also referred to as computer readable medium or machine readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

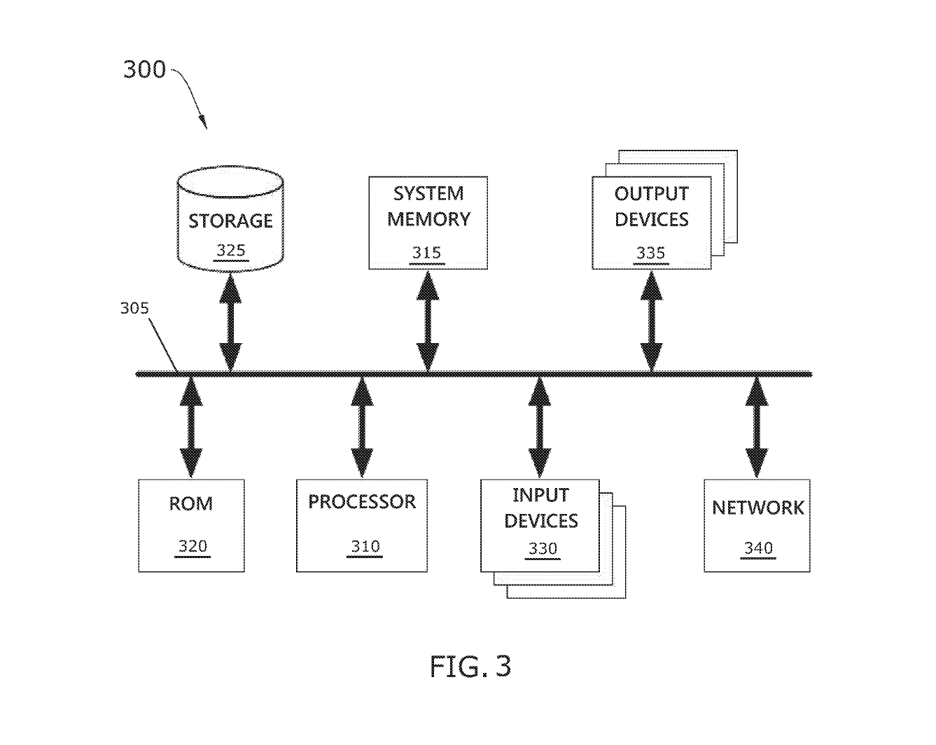

FIG. 3 conceptually illustrates an electronic system 300 with which some embodiments of the invention are implemented. The electronic system 300 may be a computer, phone (e.g., cell phone, mobile phone, smartphone, etc.), PDA (e.g., iPod, other handheld computing device, etc.), or any other sort of electronic device or computing device. Such an electronic system includes various types of computer readable media and interfaces for various other types of computer readable media. Electronic system 300 includes a bus 305, processing unit(s) 310, a system memory 315, a read-only 320, a permanent storage device 325, input devices 330, output devices 335, and a network 340.

The bus 305 collectively represents all system, peripheral, and chipset buses that communicatively connect the numerous internal devices of the electronic system 300. For instance, the bus 305 communicatively connects the processing unit(s) 310 with the read-only 320, the system memory 315, and the permanent storage device 325.

From these various memory units, the processing unit(s) 310 retrieves instructions to execute and data to process in order to execute the processes of the invention. The processing unit(s) may be a single processor or a multi-core processor in different embodiments.

The read-only-memory (ROM) 320 stores static data and instructions that are needed by the processing unit(s) 310 and other modules of the electronic system. The permanent storage device 325, on the other hand, is a read-and-write memory device. This device is a non-volatile memory unit that stores instructions and data even when the electronic system 300 is off. Some embodiments of the invention use a mass-storage device (such as a magnetic or optical disk and its corresponding disk drive) as the permanent storage device 325.

Other embodiments use a removable storage device (such as a floppy disk or a flash drive) as the permanent storage device 325. Like the permanent storage device 325, the system memory 315 is a read-and-write memory device. However, unlike storage device 325, the system memory 315 is a volatile read-and-write memory, such as a random access memory. The system memory 315 stores some of the instructions and data that the processor needs at runtime. In some embodiments, the invention's processes are stored in the system memory 315, the permanent storage device 325, and/or the read-only 320. For example, the various memory units include instructions for processing appearance alterations of displayable characters in accordance with some embodiments. From these various memory units, the processing unit(s) 310 retrieves instructions to execute and data to process in order to execute the processes of some embodiments.

The bus 305 also connects to the input and output devices 330 and 335. The input devices enable the user to communicate information and select commands to the electronic system. The input devices 330 include alphanumeric keyboards and pointing devices (also called “cursor control devices”). The output devices 335 display images generated by the electronic system 300. The output devices 335 include printers and display devices, such as cathode ray tubes (CRT) or liquid crystal displays (LCD). Some embodiments include devices such as a touchscreen that functions as both input and output devices.

Finally, as shown in FIG. 3, bus 305 also couples electronic system 300 to a network 340 through a network adapter (not shown). In this manner, the computer can be a part of a network of computers (such as a local area network (“LAN”), a wide area network (“WAN”), or an intranet), or a network of networks (such as the Internet). Any or all components of electronic system 300 may be used in conjunction with the invention.

These functions described above can be implemented in digital electronic circuitry, in computer software, firmware, or hardware. The techniques can be implemented using one or more computer program products. Programmable processors and computers can be packaged or included in mobile devices. Programmable processors and computers can be embedded or communicably connected to computing devices of a CEST MRI machine. Computing devices communicably connected to a CEST MRI with computational software installed, such as MATLAB (MathWorks), are anticipated in the invention as described herein. The processes may be performed by one or more programmable processors and by one or more set of programmable logic circuitry. General and special purpose computing and storage devices can be interconnected through communication networks, with one or more special purpose computing devices embedded in a CEST MRI machine that is communicably connected to computing devices and displays for visual output of CEST MRI imaging.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the invention has been described with reference to numerous specific details, one of ordinary skill in the art will recognize that the invention can be embodied in other specific forms without departing from the spirit of the invention. For instance, FIGS. 1 and 2 conceptually illustrate processes in which the specific operations of each process may not be performed in the exact order shown and described. Specific operations may not be performed in one continuous series of operations, and different specific operations may be performed in different embodiments. Furthermore, each process could be implemented using several sub-processes, or as part of a larger macro process. Thus, one of ordinary skill in the art would understand that the invention is not to be limited by the foregoing illustrative details, but rather is to be defined by the appended claims.

I claim:

1. A non-invasive CEST MRI imaging method for early detection of disease and mapping of disease severity comprising:
acquiring, by way of a magnetic resonance imaging (MRI) machine, a T2-image as an anatomical image;
acquiring, by way of the MRI machine, a CEST reference image at a particular Soutside chemical shift and at a signal frequency outside a range of frequency that decreases magnetization of related proteins;
acquiring, by way of the MRI machine, a plurality of CEST images at a plurality of specific Swithin chemical shifts and frequencies that decrease the magnetization of the related proteins;
calculating a difference in magnetization between Soutside and Swithin at each specific Swithin chemical shift and frequency for each image in the plurality of CEST images; and
detecting disease and mapping disease severity based on the calculated contrast differences.

2. The non-invasive CEST MRI imaging method of claim 1, wherein detecting disease and mapping disease severity comprises detecting hypoxia in cancerous and non-cancerous tissue.

3. The non-invasive CEST MRI imaging method of claim 2, wherein the difference in magnetization contrast is higher for hypoxic tumor tissue compared to non-hypoxic tumor and normal surrounding tissue as the hypoxia increase is decreased as demonstrated by a decrease in concentration of O₂ and difference in magnetization is decreased, wherein difference in magnetization can be used to identify and map the severity of hypoxia in tumor tissue, wherein difference in magnetization in non-cancer tissue is lower for hypoxic tissue compared to normal tissue by using chemical shift that decreases magnetization of amide proton with max contrast between 3.1 ppm to 4 ppm for detection of hypoxia of non-cancer tissue contrast as Swithin, wherein difference in magnetization is higher for the hypoxic in non-tumor tissue compared to normal tissue when chemical shift that decreases magnetization of amine proton with max contrast between 1 ppm to 3 ppm for detection of hypoxia for non-cancer tissue as Swithin compared to normal tissue.

4. The non-invasive CEST MRI imaging method of claim 1, wherein detecting disease and mapping disease severity comprises detecting tissue atrophy, wherein the plurality of specific Swithin chemical shifts decrease the magnetization from 10 ppm to 0.05 ppm downfield and −0.05 ppm to −10 ppm upfield.

5. The non-invasive CEST MRI imaging method of claim 4, the difference in magnetization contrast is lower for atrophy tissue compared to normal surrounding tissue as the atrophy severity increased difference in magnetization is decreased, wherein the difference in magnetization can be used to identify and map the severity of atrophy diseases, wherein the difference in magnetization is lowest for ventricles and water areas compared to surrounding normal tissue and the difference in magnetization can be used for early detection and mapping of the atrophy.

6. The non-invasive CEST MRI imaging method of claim 1, wherein detecting disease and mapping disease severity comprises distinguishing the edema from the tumor and identifying a tumor boundary from the edema by determining where the difference in magnetization between Soutside and Swithin is the lowest value over the plurality of plurality of specific Swithin chemical shifts.

7. The non-invasive CEST MRI imaging method of claim 6, wherein the difference in magnetization contrast is lower to the edema compared to the tumor, wherein the difference in magnetization is increased when moving away from the tumor boundary toward the tumor core, wherein the difference in magnetization for tumor tissue is higher than the difference in magnetization for a normal surrounding tissue, wherein the difference in magnetization can be used for early detection and mapping of the tumor, wherein the difference in magnetization can precisely determine the tumor boundary.

8. The non-invasive CEST MRI imaging method of claim 1, wherein detecting disease and mapping disease severity comprises distinguishing a higher-grade tumor from a lower-grade tumor to detecting lower grade tumor.

9. The non-invasive CEST MRI imaging method of claim 8, wherein the difference in magnetization contrast can be used to detect lower grade tumor that cannot be detected in another way, wherein the difference in magnetization contrast can be used to distinguish the higher-grade tumor from the lower-grade tumor, wherein the difference in magnetization is increased as the tumor grade is increased, wherein the difference in magnetization is increased as the aggressiveness of tumor increases by using chemical shift that decreases magnetization of amide proton with max contrast between 3.1 ppm to 4 ppm as Swithin.

10. The non-invasive CEST MRI imaging method of claim 1, wherein detecting disease and mapping disease severity comprises monitoring tumor response to treatment.

11. The non-invasive CEST MRI imaging method of claim 10, wherein the difference in magnetization contrast can be used to monitor response of tumor to the treatment, wherein the difference in magnetization is decreased in most cancer types after response of cancer to the treatment by using chemical shift that decreases magnetization of amide proton with max contrast between 3.1 ppm to 4 ppm as Swithin, wherein the difference in magnetization is increased after response of cancer to the treatment by using chemical shift that decreases magnetization of amine proton with max contrast between 1 ppm to 3 ppm as Swithin.

12. The non-invasive CEST MRI imaging method of claim 1, wherein endogenous protons of tissues can be used as an endogenous contrast to detect amyloid beta plaques, aggregation proteins, and neurofibrillary tangles in neurodegenerative diseases.

13. The non-invasive CEST MRI imaging method of claim 1, wherein endogenous protons of tissues can be used as an endogenous contrast to detect the hypoxia in cancer and non-cancer tissue.

14. The non-invasive CEST MRI imaging method of claim 1, wherein endogenous protons of tissues can be used as an endogenous contrast to detect the atrophy.

15. The non-invasive CEST MRI imaging method of claim 1, wherein endogenous protons of tissues can be used as an endogenous contrast to detect and distinguish one of the edema and the water from the tumor and determine tumor boundary.

16. The non-invasive CEST MRI imaging method of claim 1, wherein endogenous protons of tissues can be used as an endogenous contrast to detect the lower grade tumor and can distinguish the higher-grade tumor from the lower-grade tumor.

17. The non-invasive CEST MRI imaging method of claim 1, wherein endogenous protons of tissues can be used as an endogenous contrast to monitor the response of cancer to treatments.

18. The non-invasive CEST MRI imaging method of claim 1, wherein a decrease in magnetization of cancer proteins starts from chemical shift 0.05 ppm to 10 ppm downfield and from −0.05 ppm to −10 ppm upfield and the maximum decrease of the magnetization of cancer proteins is approximately between 1 ppm and 5 ppm downfield and approximately between −1 ppm to −5 ppm upfield.

19. The non-invasive CEST MRI imaging method of claim 1, wherein decrease magnetization of amyloid beta, neurofibrillary tangles, and aggregation proteins starts from chemical shift 0.05 ppm to 10 ppm downfield and from −0.05 ppm to −10 ppm upfield and the maximum decrease of the magnetization of amyloid beta, neurofibrillary tangles and aggregation proteins is approximately between 1 ppm and 5 ppm from water downfield and approximately between −1 ppm to −5 ppm from water upfield.

20. The non-invasive CEST MRI imaging method of claim 1, wherein the difference in magnetization between Soutside and Swithin contrast is higher for accumulation amyloid beta plaques, aggregation proteins and neurofibrillary tangles compared to normal surrounding tissue as the accumulation, wherein when the concentration of proteins of the difference in magnetization between Soutside and Swithin is increased, then the difference in magnetization between Soutside and Swithin can be used to identify and map the severity of neurodegenerative diseases and used for early detection and mapping of diseases.
